(19) United States
(12) Patent Application Publication
(10) Pub. No.: US 2013/0092526 A1
(43) Pub. Date: Apr. 18, 2013

(54) Method of activation of noble metal for measurement of glucose and associated biosensor electrode

(71) Applicant: CPFilms Inc., Fieldale, VA (US)

(72) Inventors: Sarkhadoun Yadegar, Reseda, CA (US); Mohtashim Saif, Chino, CA (US); Shiwei Liu, Stevenson Ranch, CA (US); Hassan Memarian, Porter Ranch, CA (US)

(73) Assignee: CPFilms Inc., Fieldale, VA (US)

(21) Appl. No.: 13/573,687

(22) Filed: Oct. 3, 2012

Related U.S. Application Data
(60) Provisional application No. 61/542,678, filed on Oct. 3, 2011.

(51) Int. Cl. C12Q 1/00

(52) U.S. Cl. CPC C12Q 1/006; B82Y 15/00
USPC 204/192.1; 204/403.04; 977/755

(57) ABSTRACT

An electrochemical glucose biosensor comprising two electrodes with at least one of electrodes having both a metallic layer and a non-metallic layer in direct contact with the metallic layer. The metallic layer is comprised of a noble metal element. A glucose reactive strip connects the first electrode and the second electrode.

### CROSS REFERENCE TO RELATED APPLICATION(S)

This application claims the benefit of U.S. Provisional Patent Application Ser. No. 61/542,678, filed Oct. 3, 2011, the entire disclosure of which is incorporated by reference herein.

### BACKGROUND OF THE INVENTION

1. Field of the Invention

This disclosure is related to the field of reactivity of noble metals with analytes, and specifically to biosensors which are designed to react with glucose and the methods of making the same.

2. Description of Related Art

The human body in its functions requires a very delicate balance of chemicals in order to process, transfer, and metabolize. The levels of key metabolites in a human body are only properly maintained within a known physiological range by normal action of body organisms. Any repetitive change in the concentration of these known chemicals from these expected ranges is generally a sign of illness.

In today's society where there is a lack of exercise and also the unhealthy diet of processed food has replaced the normal healthy food of many households, a very common illness which is rapidly on the rise is diabetes. Diabetes is simply characterized by elevated blood glucose concentration as a result of insufficient (or no) insulin production in the pancreas or, in some cases, by insulin resistance. In effect, the body's blood chemistry is regularly outside the normal range of such chemistry.

According to the International Diabetes Federation, there are currently 246 million diabetics worldwide and the number is expected to reach 380 million by 2025. In many cases, even chronic diabetes is a very controllable disease so long as the body's functions are correctly monitored and glucose (or insulin) is supplied to the body when appropriate. However, in order to correctly respond to the body's needs, the body's needs must be determined. In order to determine their current blood chemistry, diabetics will regularly take samples of their blood and perform chemical analysis to determine if they have an excess (or lack) of glucose. If an excess of glucose is detected, insulin can be added to the patient's blood stream (by specific injection, through installed pumps, or through other means) to attempt to return the glucose level to within a normal range. Similarly, if blood glucose is too low, the individual can consume carbohydrates to again return the level to the normal range.

While diabetes is generally considered controllable because the blood chemistry can be regulated externally, the effects of uncontrolled diabetes, even for short periods of time, can be catastrophic. Diabetes generally damages blood vessels and nerves and therefore many diabetics can suffer injuries which they do not realize are present, until they have become highly progressed and can require aggressive and life altering treatment such as amputation of limbs. Failure to control diabetes can also result in even more major complications including kidney failure, blindness, brain damage, heart attack, or death. As should be apparent from the above, a prerequisite for such tight control at all times is accurate and frequent monitoring of the blood glucose level as that informs the treatment plan (i.e., dosage of insulin) and provides that any imbalance can be promptly corrected with minimal resultant damage.

Because of the need to regularly monitor blood sugar, there is a very high demand for products that can accurately determine the amount of glucose in blood. These devices are generally called glucometers and most modern ones operate by having the person take a drop of blood (often obtained from a fingertip) and place it on a strip which is reactive to glucose. The amount of reaction is then detected and the glucometer returns an indication of blood glucose level. The diabetic can then consume food or utilize insulin to correct an imbalance. As should be apparent, the need to test blood multiple times a day with single use biosensor strips results in a large demand for these strips.

Single-use glucose biosensors in the form of test strips generally follow a similar pattern of construction. Test strips generally contain an enzyme (often glucose oxidase or glucose dehydrogenase), mediator (often ferricyanide), indicator, and many additional ingredients in the form of dry layers placed on a substrate. These dry layers may then be in chemical contact with two or more electrodes, which are connected to electrical connections in the glucometer.

The methods of measurement of the tests strips are generally photometry and electrochemistry. Both methods use similar designs of the detection zone. In a photometric system, measurement is done by illumination of the reacted sample with light, using a narrow wavelength bundle from a LED. A part of the diffuse reflection arrives at a photodetector and is converted to a current.

In an amperometric system, which is generally more common, measurements convert the reaction product to an oxidized form again. This reaction occurs at the surface of electrodes on the test strip, and diffusion is needed to transport reduced species to the surface and oxidized mediator away from it. In principle this is a slower process than the instantaneous observation in photometry. However, the reaction can be finished in a few seconds or less and is often more robust and simpler to implement.

Electrochemical measurement quality is a result of a close cooperation between dry chemistry and measurement method including the evaluation algorithm. The design of electrochemical test strips can vary significantly, especially in the number of electrodes used to contact the reagent and blood sample and in the way in which a measurement is taken from the strip. However, most test strips utilize the same general principles even within design constraints imposed by the glucometer's specific design.

Generally, in the test strips the enzyme (glucose oxidase) catalyzes the oxidation of glucose to gluconolactone and a redox cofactor. The gluconolactone is then hydrolyzed to gluconic acid. The hydrolyzation serves to transfer electrons from the glucose to the mediator (ferricyanide), and a known amount of the indicator (ferrocyanide) is created per each molecule of glucose so transferred. The reduction of the ferricyanide to ferrocyanide in the reagent layer results in the re-oxidation of the enzyme.

In any case the measured current and therefore calibration with regards to the glucose level is proportional to the working electrode area in the test strip. The working electrode is the electrode where an electrochemical reaction takes place that is proportional to the amount of glucose in the blood.

The counter electrode must function properly so that the working electrode can accurately measure the level of glucose in the blood. In effect, an opposing reaction must occur at the counter electrode for the reaction at the working electrode to continue. The electrodes are generally positioned on one or more substrates which are then combined with the dry ingredients (possibly with additional substrates) to form the test strip. FIG. 1A shows a block diagram of how the reactions occur and the electrochemical measurement of the glucose level based on the consumed concentration of the ferrocyanide produced during the reduction of ferricyanide.

After electrode potential is applied on the system the current is measured. The counter electrode potential is defined by the ratio of ferricyanide and ferrocyanide at the electrode surface. The applied potential provides a diffusion-limited current at the working electrode, so the ferrocyanide concentration may be determined by biamperometry. The meter measures working electrode current, that is proportional to ferrocyanide concentration and hence is in direct relation to the glucose concentration. Utilizing an algorithm which is generally specific to the test strip and glucometer, the glucose concentration can then be determined and returned to the user.

One advantage of this method of measurement is that both electrodes in the construction of the test strip can be the same metal, which simplifies construction. Specifically, electrode and substrate constructs may be formed in large rolls or other structures which can then be cut apart into the desired construct for use in the test strips. Effectively, the selection of which is the working vs. counter electrode is determined simply by placement in the resultant test strip with the two electrodes being effectively identical, in an embodiment.

The mediator, ferricyanide, needs to react equally well at both electrodes. Hence it's advantageous to use the same material (and construction) for both electrodes to simplify construction. Most of the current glucometers use plastic substrates coated with gold or other noble metals, generally in the purest form commercially feasible, to function as the electrode.

The quality of the coating on the substrate and its activity as to the transfer of the electron from the mediator to the surface of the electrode and the diffusion rate of the ferrocyanide onto the working electrode play an important role in the accurate measurement of the glucose level in blood. Specifically, the ability to accurately diffuse the ferrocyanide onto the electrode results in an accurate count of the number of ferrocyanide molecules, and thus the amount of glucose. The more accurate the count, the better the sensor's sensitivity at the electrode.

Further, as the glucometer does not directly measure the number of molecules, but measures their electrochemical properties, it is also important that the electrical properties of the strips have a consistent relationship between measured electrical potential and glucose concentration. In many respects, the consistency of the reading is just as important, if not more so, as the raw sensitivity of the reading when the algorithm makes the determination of glucose concentration. A consistent reading of potential will provide a more consistent (and thus more accurate) result, even if sensitivity is reduced.

One concern with regards to the consistency of readings with the test strips is the aging of raw (or completed) test strip products or components. As the strips are designed to be chemically reactive, over time material used in the strips can alter in chemical composition, which can in turn alter its electrical properties, and thus alter the resultant output determination. As should be appreciated, it is generally not possible for the meter to take the age of the strip into account (or even determine it) and it is generally impossible for a test strip manufacturer to accurately control product shelf storage either at stores or by end users except to discourage use of substantially old strips (e.g. via printed expiration dates). Further, it is inefficient to be forced to shelve stock for a waiting period before using it to make sure that it has aged to a consistent level before being sold.

With regards to some pure (or nearly pure) noble metal electrodes (e.g., palladium), over time it is believed that the metal will oxidize which will alter the electrical conductive properties. Experimental testing has revealed that such modification occurs primarily in the first 100 days. Because it is generally easier to calibrate a glucometer to accept a consistently aged product (as opposed to a consistently new product) for commercial reasons and to deal with compliance issues by end users, it is desirable to produce a test strip where the electrodes behave in the same fashion as they do after aging (specifically after aging at least 100 days) at the time of production without them showing additional age related effects (effectively they are “pre-aged”). This allows for both new and aged products to perform at the same, consistent, level improving product shelf life and consistency of readings to the end user.

### SUMMARY

Because of these and other problems in the art, described herein, among other things, is an electrochemical glucose biosensor comprising: a first electrode comprising a non-conducting and chemically inert substrate having disposed thereon an electrically conductive layer; a second electrode comprising a non-conducting and chemically inert substrate having disposed thereon an electrically conductive layer, the electrically conductive layer of the second electrode comprising a metallic layer comprising a noble metal element; and a glucose reactive strip connecting the first electrode and the second electrode. The electrically conductive layer of the first electrode comprises a metallic layer comprising a noble metal element; and a non-metallic electrically conductive layer disposed in direct contact with the metallic layer.

In some embodiments, the noble metal element of the first electrode is palladium.

The thickness of the metallic layer comprising noble metal of both the first and second electrodes varies. In some embodiments, the thickness is between about 10 nanometers and about 10 microns. In other embodiments, the thickness is between about 10 nanometers and about 50 nanometers. In still other embodiments, the thickness is between about 20 nanometers and about 30 nanometers.

The thickness of the non-metallic electrically conductive layer can also vary. In some embodiments, the thickness is between about 1 nanometer and about 10 nanometers.

The glucose reactive strip may comprise an enzyme, a mediator, and an indicator. In some embodiments, the enzyme is glucose oxidase. The mediator also may be ferricyanide.

In another embodiment, the electrically conducting layer of the second electrode may further comprise a non-metallic electrically conductive layer disposed in direct contact with the metallic layer. In such an embodiment, the non-metallic electrically conductive layers of both the first and second electrode may comprise carbon.

In some embodiments, the noble metal element of the second electrode comprises gold. In other embodiments, the non-metallic electrically conductive layer comprises carbon. In still other embodiments, the substrate may comprise a thermoplastic polymer.

Also disclosed herein is a method of forming an activated electrochemical glucose biosensor comprising: providing a substrate; sputtering a first layer over the substrate, the first layer comprising either a metallic layer comprising a noble metal element or a non-metallic electrically conductive layer; and sputtering a second layer over the first layer, the second layer comprising either a metallic layer comprising a noble metal element or a non-metallic electrically conductive layer. In this method, the second layer is a metallic layer comprising a noble metal element if the first layer is a non-metallic electrically conductive layer; and the second layer is a non-metallic electrically conductive layer if the first layer is a metallic layer comprising a noble metal element.

In some embodiments, the first and second sputtering steps occur in a vacuum. In other embodiments, the noble metal element is palladium.

Also disclosed herein is an electrochemical glucose biosensor comprising: a first electrode comprising an electrically conductive layer, a second electrode comprising an electrically conductive layer, the electrically conductive layer comprising a metallic layer comprising a noble metal element; and a glucose reactive strip connecting the first electrode and the second electrode. In this embodiment, the electrically conductive layer of the first electrode comprises: a metallic layer comprising palladium; and a non-metallic electrically conductive layer disposed in direct contact with the metallic layer. In some embodiments, the noble metal element of the second electrode comprises gold. In other embodiments, the non-metallic electrically conductive layer comprises carbon and the thickness of the non-metallic electrically conductive layer is between about 1 nanometer and about 10 nanometers.

### BRIEF DESCRIPTION OF THE DRAWINGS

FIG. 1 shows a functional block diagram of the chemical action and operation of a test strip in a glucometer.

FIGS. 2A-2D show cut-through diagrams of different embodiments of electrodes of the present invention.

FIG. 3 shows a functional block diagram of a roll-to-roll sputtering chamber which may be used to create electrodes of the present invention.

FIG. 4 shows a cut-through diagram of an embodiment of an electrode after deposition of the metallic layer.

FIG. 5 shows a cut-through diagram of an embodiment of an electrode after deposition of a non-metallic layer on the metallic layer.

FIG. 6 provides for graphical results from an EIS analysis for electrodes comprising palladium with carbon, an aged palladium electrode without carbon, and a new palladium electrode without carbon.

FIG. 7 shows a graphical CV comparison of the palladium/carbon and aged palladium samples further indicating similar characteristics.

FIG. 8 shows a block diagram of a test strip including electrodes of an embodiment of the present invention.

### DETAILED DESCRIPTION OF PREFERRED EMBODIMENTS

Described herein is a method of producing and activating a layer of noble metal(s) which can be used as an electrode in diabetic test strips, for other forms of glucose monitoring, or for other applications where such activation is desirable. “Activating” as that term is used herein, comprises effectively making the electrode perform as if it has aged a particular amount of time, without such aging actually occurring. Thus, an activated product would be one which performs similarly when it is initially produced (a “new” product) to a product which has been allowed to sit under standard shelving and packaging conditions for a period of time (an “aged” product). It should be noted that certain packaging, manufacturing, and other techniques may be used to hinder or reduce the effect of aging on a product. The present disclosure, however, is not concerned with these as the desire is to produce a product which has known and consistent “pre-aged” characteristics at the time it is new to effectively hinder the product from changes due to further aging.

The concept of activating is designed to effectively take age effects on electrodes into account and move the product initially down an age curve. Reactivity of some noble metal electrodes (depending on the noble metal and without any activation having been performed) is generally more pronounced immediately after production, and the amount of change reduces over time. More specifically, for some noble metal electrodes (e.g., palladium) changes have been noticed to be significantly more pronounced in the first 100 days after manufacture than in the time period of longer than 100 days. Further, significantly longer aged products can often be eliminated from use by compliance with printed product expiration dates. Thus, in an embodiment of the present disclosure, an “aged” product comprises an electrode of a type known to those of ordinary skill which has been left idle for 100 days under standard storage conditions for such product. A “new” product comprises a product at the time it is packaged and initially placed in such conditions. An “activated” product is a product which, when new, mimics the characteristics of a non-activated product which has been aged.

One thing to note is that the electrode contemplated herein behaves similarly to a traditional electrode which has aged and which has, at least partially, oxidized. However, the processes herein do not serve to oxidize the noble metal. Instead, some of the electrodes discussed herein have the noble metal first deposited, and then coated with a non-metallic layer which, without being limited to any particular mode of operation, causes the electrode to mimic an aged electrode without having to create oxidation. However, the reactivity of some noble metals (e.g., gold, silver, etc.) generally remains the same over time, and thus, there is no need for a coated non-metallic layer.

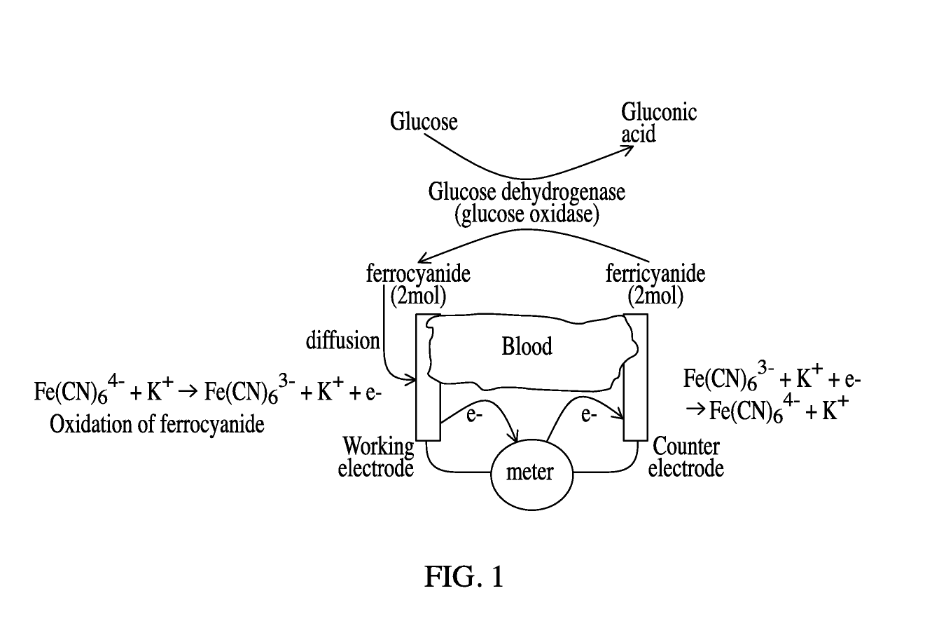

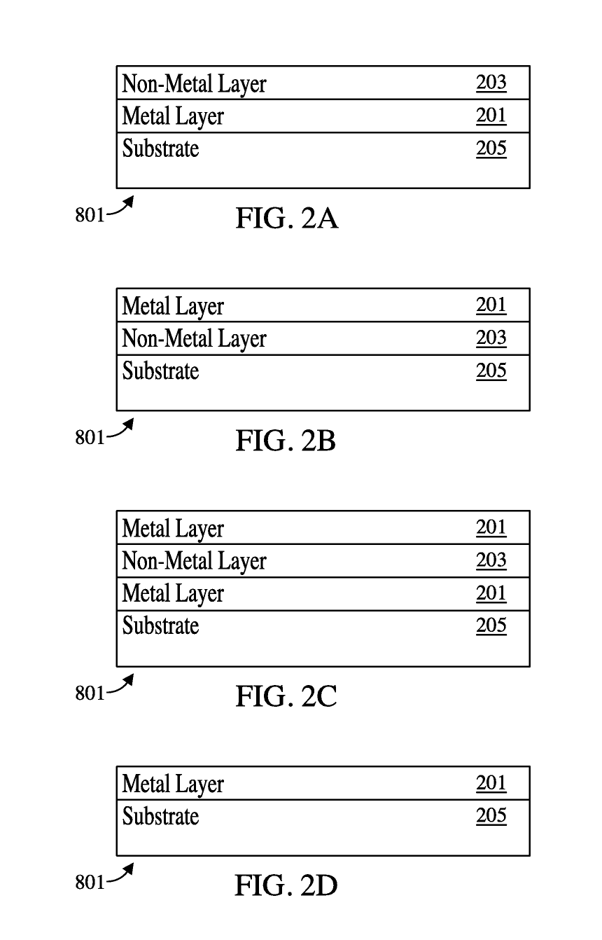

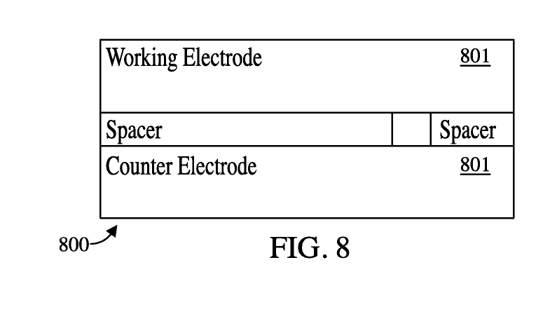

FIG. 2 provides a cut through drawing of various electrodes (801) as may be used as a part of a test strip. In particular, these electrodes may comprise either of the working or the counter electrode of a strip as shown in FIGS. 1 and 8. As shown in FIGS. 2A-2C, the electrode (801) includes both metallic layer(s) (201) and non-metallic layer(s) (203). The metallic layer(s) (201) and non-metallic layer(s) (203) are deposited on the substrate (205) in either order of metallic/non-metallic (shown in FIG. 2A) non-metallic/metallic (shown in FIG. 2B), or metallic/non-metallic/metallic (shown in FIG. 2C) in order to produce the highest activity of the electrode material. It should be recognized that additional metallic or non-metallic layers and arrangements may alternatively be used to produce alternative arrangements depending on the desired operation and characteristics of the electrode (e.g., simulated aging time). As shown in FIG. 2D, it may be preferable for some electrodes (801) (e.g., those comprising gold) to omit the non-metallic layer.

Generally a test strip (800) will comprise at least two electrodes (801) and will generally comprise a substrate onto which the chemical ingredients (such as the enzyme and mediator) will also be deposited. This may comprise substrate (205) with the chemicals deposited at a different location from those pictured in FIG. 2, or may comprise a different substrate which serves as the base for the chemical actions and to which substrate (205) is attached depending on the test strip design. Further, each strip (800) may include at least two relatively identical electrodes (801) (specifically, a counter electrode and a working electrode both comprised of the same noble metal, e.g., palladium) or at least two different electrodes (specifically, a counter electrode comprised of, e.g., palladium, and a working electrode comprised of, e.g., gold). Additionally, the strip (800) may comprise more than two electrodes, two or more electrodes with different shapes, or two or more electrodes of different constructions, depending on the design of the glucometer the strip (800) is designed to operate with. An embodiment of a test strip is shown in FIG. 8.

The substrate (205) (and any other substrate, if present) may comprise any material, either flexible or rigid, which is generally non-conducting and chemically inert to the contemplated reactions discussed above. This will often be a form of plastic and can include, but is not limited to, polyester, polyethylene, polycarbonate, polypropylene, nylon or other polymers.

Generally the metallic layer (201) will comprise a noble metal. Specific examples of noble metals include, but are not limited to, ruthenium, rhodium, palladium, silver, osmium, iridium, platinum, gold or any combination thereof. In some embodiments, the metal will be provided in the purest form commercially practicable at the time of deposition so as to eliminate oxidized forms. The metal may be deposited on a flexible substrate through different known deposition techniques as a very thin layer, generally ranging from about 10 nanometers to about 10 micron, preferably between about 10 nanometers and about 50 nanometers, more preferably between about 20 nanometers and about 30 nanometers. The metallic layer may be activated by a non-metallic electrically conductive layer which is deposited on the surface of the noble metal as it was deposited on the substrate. As noted above, however, in some instances, it may be unnecessary to activate the metallic layer with the deposition of the non-metallic layer.

If present, the non-metallic layer will generally comprise any conductive non-metallic material known to those of ordinary skill. This includes, but is not limited to, materials of conductive transparent coatings such as, but not limited to, carbon (often in form of graphite) and conductive polymers, although other conductive non-metallic materials and combinations thereof may be used in other embodiments. The non-metallic conductive layer will generally comprise a much thinner layer compared to the metallic layer and will usually range from 1 nanometer to 10 nanometers for a palladium electrode ranging from 15 nanometers to 100 nanometers.

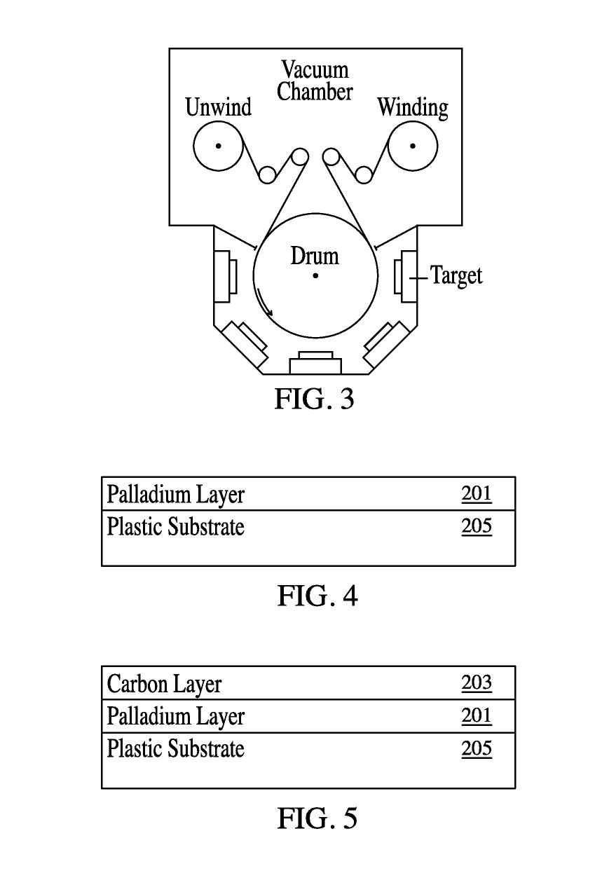

FIG. 3 shows an embodiment of a roll-to-roll coating system which can be used, in an embodiment, for sputtering as a method of controlled deposition of both the metallic and non-metallic layers on a plastic substrate. Such a roll-to-roll coating system generally produces an atomic level coating on the plastic film in a roll-to-roll format, hence reducing the cost of the material and is a preferred embodiment for electrode construction. The roll can then be cut apart to form the specific electrode parts of each test strip in a known fashion. Sputtering application generally provides a very clean and homogenous layer with little to no contamination by environmental factors, as any or all layers can be deposited in a vacuum or an inert environment to provide purer coatings. However, one of ordinary skill would understand that sputtering is not necessary and other methods of deposition may be utilized in electrode construction.

FIGS. 4 and 5 show an arrangement of the material formed by the device of FIG. 3 operated according to known methods. In FIG. 4 the initial metallic layer has been deposited in a first sputtering step. In FIG. 5 the non-metallic layer has been placed thereon via a second sputtering step. The metallic layer(s) is constructed by using any of the noble metals, however, in the embodiment of FIG. 4 it comprises palladium. The layer will preferably be highly uniform and generally of consistent thickness. In FIG. 5, this metallic layer(s) has had placed thereon a non-metallic layer(s) (in this case carbon). The non-metallic layer(s) is deposited by the same method of sputtering at a controlled thickness, using non-metallic conductive materials for deposition. In FIG. 5 the layer of non-metallic material is no more than 1 micron thick, preferably between 1 angstrom and 1 micron and more preferably from 5 angstroms to 50 nanometers thick. However, this is by no means required and other thicknesses may be used.

An electrode including layered palladium and carbon has been determined to be more resistive to age related alteration of its electrical properties than an electrode which does not include the non-metallic layer and to provide electrical properties similar to an aged (100 days) plain palladium electrode. That is, such a construct is activated as discussed above.

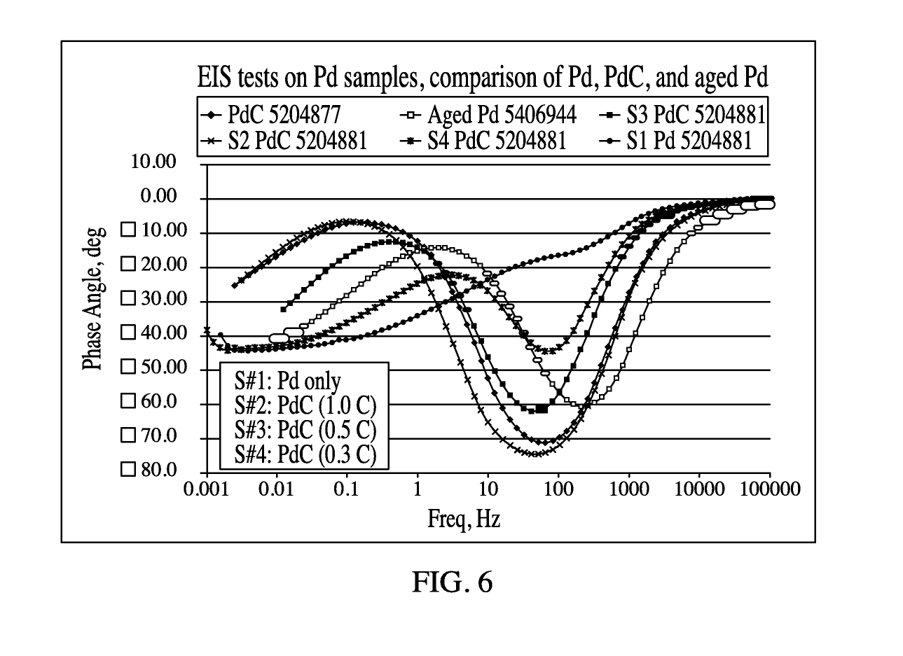

FIG. 6 provides for an EIS analysis showing the difference in a new sample (less than 72 hours old) palladium (around 28 nanometers) with a carbon layer electrode (three samples with different thicknesses of the carbon layer), vs. an aged (150 days old) palladium (around 26-28 nanometers) layer without carbon and a new (less than 72 hours old) palladium (28 nanometers) layer without carbon. As can be seen, the unaged palladium/carbon layered electrode (with a 0.5 micron thickness carbon layer) fairly closely tracks performance of the aged palladium electrode.

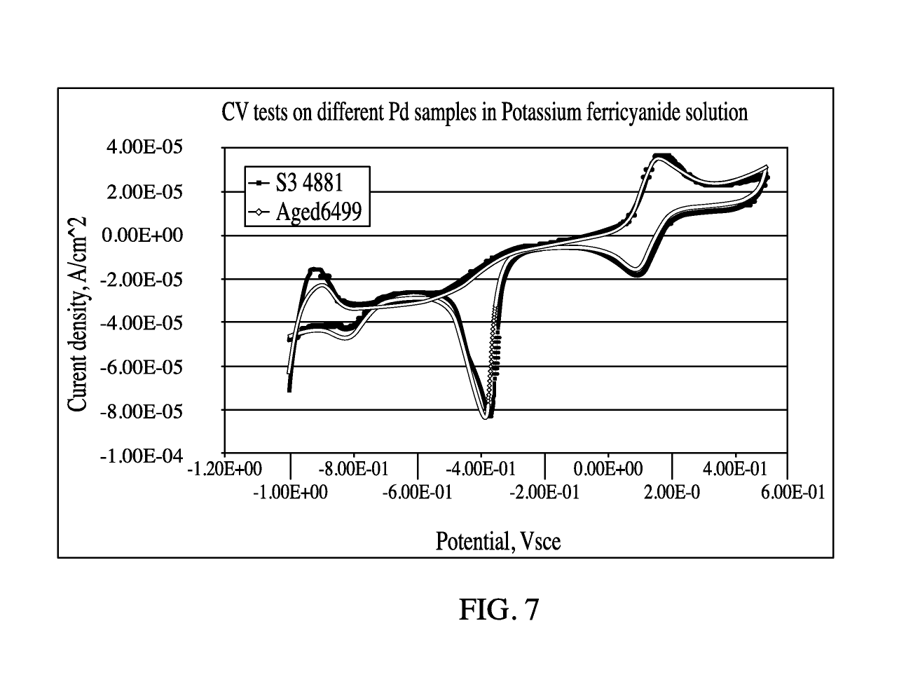

FIG. 7 shows a CV comparison of the same palladium/carbon and aged palladium samples further indicating similar characteristics.

As should be apparent from FIG. 6, not only can an electrode of specific age be “simulated” by the use of a carbon layer, altering the thickness of the carbon layer can allow for slightly different properties to be obtained.

While the inventions have been disclosed in conjunction with a description of certain embodiments, including those that are currently believed to be the preferred embodiments, the detailed description is intended to be illustrative and should not be understood to limit the scope of the present disclosure. As would be understood by one of ordinary skill in the art, embodiments other than those described in detail herein are encompassed by the present invention. Modifications and variations of the described embodiments may be made without departing from the spirit and scope of any invention herein disclosed.

It will further be understood that any of the ranges, values, or characteristics given for any single component of the present disclosure can be used interchangeably with any ranges, values or characteristics given for any of the other components of the disclosure, where compatible, to form an embodiment having defined values for each of the components, as given herein throughout. Further, ranges provided for a genus or a category can also be applied to species within the genus or members of the category unless otherwise noted.

1. An electrochemical glucose biosensor comprising:
a first electrode comprising a non-conducting and chemically inert substrate having disposed thereon an electrically conductive layer, the electrically conductive layer comprising:
a metallic layer comprising a noble metal element; and
a non-metallic electrically conductive layer disposed in direct contact with the metallic layer;
a second electrode comprising a non-conducting and chemically inert substrate having disposed thereon an electrically conductive layer, the electrically conductive layer comprising a metallic layer comprising a noble metal element; and a glucose reactive strip connecting the first electrode and the second electrode.

2. The electrochemical glucose biosensor of claim 1 wherein the noble metal element of the first electrode is palladium.

3. The electrochemical glucose biosensor of claim 2 wherein the thickness of the metallic layer comprising noble metal of both the first and second electrodes is between about 10 nanometers and about 10 microns.

4. The electrochemical glucose biosensor of claim 2 wherein the thickness of the metallic layer comprising noble metal of both the first and second electrodes is between about 10 nanometers and about 50 nanometers.

5. The electrochemical glucose biosensor of claim 2 wherein the thickness of the metallic layer comprising noble metal of both the first and second electrodes is between about 20 nanometers and about 30 nanometers.

6. The electrochemical glucose biosensor of claim 2 wherein the thickness of the non-metallic electrically conductive layer is between about 1 nanometer and about 10 nanometers.

7. The electrochemical glucose biosensor of claim 2 wherein the glucose reactive strip comprises an enzyme, a mediator, and an indicator.

8. The electrochemical glucose biosensor of claim 7 wherein the enzyme is glucose oxidase.

9. The electrochemical glucose biosensor of claim 8 wherein the mediator is ferricyanide.

10. The electrochemical glucose biosensor of claim 2 wherein the electrically conducting layer of the second electrode further comprises a non-metallic electrically conductive layer disposed in direct contact with the metallic layer.

11. The electrochemical glucose biosensor of claim 10 wherein the non-metallic electrically conductive layers of both the first and second electrode comprise carbon.

12. The electrochemical glucose biosensor of claim 2 wherein the noble metal element of the second electrode comprises gold.

13. The electrochemical glucose biosensor of claim 12 wherein the non-metallic electrically conductive layer comprises carbon.

14. The electrochemical glucose biosensor of claim 2 wherein the non-metallic electrically conductive layer comprises carbon.

15. The electrochemical glucose biosensor of claim 2 wherein the substrate comprises a thermoplastic polymer.

16. A method of forming an activated electrochemical glucose biosensor comprising:
providing a substrate;
sputtering a first layer over the substrate, the first layer comprising either a metallic layer comprising a noble metal element or a non-metallic electrically conductive layer; and
sputtering a second layer over the first layer, the second layer comprising either a metallic layer comprising a noble metal element or a non-metallic electrically conductive layer and wherein:
the second layer is a metallic layer comprising a noble metal element if the first layer is a non-metallic electrically conductive layer; and
the second layer is a non-metallic electrically conductive layer if the first layer is a metallic layer comprising a noble metal element.

17. The method of claim 16 wherein the first and second sputtering steps occur in a vacuum.

18. The method of claim 16 wherein the noble metal element is palladium.

19. An electrochemical glucose biosensor comprising:
a first electrode comprising an electrically conductive layer, the electrically conductive layer comprising:
a metallic layer comprising palladium; and
a non-metallic electrically conductive layer disposed in direct contact with the metallic layer;
a second electrode comprising an electrically conductive layer, the electrically conductive layer comprising a metallic layer comprising a noble metal element; and
a glucose reactive strip connecting the first electrode and the second electrode.

20. The electrochemical glucose biosensor of claim 19 wherein the noble metal element of the second electrode comprises gold.

21. The electrochemical glucose biosensor of claim 19 wherein the non-metallic electrically conductive layer comprises carbon and the thickness of the non-metallic electrically conductive layer is between about 1 nanometer and about 10 nanometers.
